(19) United States
(12) Patent Application Publication
(10) Pub. No.: US 2025/0367260 A1
(43) Pub. Date: Dec. 4, 2025

(54) TIRZEPATIDE COMPOSITIONS AND PREPARATION METHOD

(71) Applicant: Galenicum Health S.L.U., Esplugues de Llobregat (ES)

(72) Inventors: Luis Gómez COELLO, San Agustín de Guadalix (ES); Jamie Osuna LÓPEZ, Madrid (ES)

(21) Appl. No.: 19/214,902

(22) Filed: May 21, 2025

Related U.S. Application Data
(63) Continuation-in-part of application No. 19/019,721, filed on Jan. 14, 2025.

(30) Foreign Application Priority Data
May 31, 2024 (EP) 24382587
Feb. 7, 2025 (EP) 25382104

(51) Int. Cl. A61K 38/22; A61K 47/18; A61K 47/22

(52) U.S. Cl. CPC A61K 38/2235; A61K 47/18; A61K 47/22

(57) ABSTRACT

Disclosed herein is a stable pharmaceutical composition comprising a dual Glucose-Dependent Insulinotropic Polypeptide (GIP) and Glucagon-Like Peptide-1 (GLP-1) receptor agonist (e.g., tirzepatide). Also disclosed herein is an aqueous composition comprising tirzepatide and at least one non-inorganic buffer; formulated for enhanced storage stability and reduced aggregation, ensuring optimal therapeutic efficacy in the treatment of diabetes mellitus and obesity.

### RELATED APPLICATIONS

This application is a continuation-in-part of U.S. patent application Ser. No. 19/019,721, filed on Jan. 14, 2025, and this application claims priority to European patent application EP24382587, filed on May 31, 2024 and European patent application EP25382104, filed on Feb. 7, 2025.

### FIELD

Disclosed herein is a stable pharmaceutical composition comprising a dual Glucose-Dependent Insulinotropic Polypeptide (GIP) and Glucagon-Like Peptide-1 (GLP-1) receptor agonist (e.g., tirzepatide). Also disclosed herein is an aqueous composition comprising tirzepatide and at least one non-inorganic buffer; formulated for enhanced storage stability and reduced aggregation, ensuring optimal therapeutic efficacy in the treatment of diabetes mellitus and obesity.

### BACKGROUND

The development of pharmaceutical compositions comprising therapeutic peptides, such as tirzepatide, is of significant interest due to their potential in treating conditions like diabetes and obesity. Tirzepatide, as a dual GIP/GLP-1 receptor agonist, offers improved glycemic control and weight reduction benefits over traditional GLP-1 analogues. However, like other peptide-based pharmaceuticals, tirzepatide is susceptible to degradation, requiring careful formulation to ensure stability, efficacy, and safety. These peptides are prone to chemical instability, such as oxidation, hydrolysis, and deamidation, which may affect their structural integrity and therapeutic function. Additionally, physical instability, such as aggregation or precipitation, may lead to reduced bioavailability and potential immunogenic responses. Developing a stable tirzepatide formulation is, therefore, a key challenge in pharmaceutical sciences.

Inorganic buffers such as dibasic sodium phosphate are used in commercial formulations of tirzepatide. EP3810201 and WO2024006662 disclose pharmaceutical compositions of tirzepatide comprising sodium phosphate dibasic heptahydrate as a buffering agent.

While inorganic buffers like sodium phosphate offer effective pH control, they may also present certain disadvantages in pharmaceutical formulations. These include potential incompatibility with biologically active peptides, leading to precipitation, hydrolysis, or unwanted interactions that reduce drug stability. Additionally, sodium phosphate buffers can exacerbate injection site irritation in some patients due to their ionic nature. Another challenge is their pH-dependent solubility, which can lead to crystallization under certain storage conditions, thereby compromising the formulation's stability and efficacy. Furthermore, phosphate buffers have been associated with calcium chelation, potentially leading to precipitate formation in the presence of divalent cations. Given these drawbacks, alternative buffering agents are desirable for improving the stability, safety, and tolerability of tirzepatide formulations.

Another challenge is the avoidance of fibril formation, a process by which these peptides tend to form well-ordered, thread-like macromolecular aggregates, poses a significant challenge in GLP-1 analogues formulation. In the literature, heat treatment of GLP-1 solutions has been suggested for increasing the shelf life and stability of the pharmaceutical solutions due to fibril formation.

Fibrils in parenteral compositions present several significant disadvantages that impact the safety, efficacy, and regulatory compliance of pharmaceutical products. Immunogenicity is a major concern, as fibrils can provoke an immune response, potentially leading to adverse reactions in patients, ranging from mild allergic responses to severe immunological complications. The presence of fibrils also signifies a loss of therapeutic efficacy, as aggregated proteins are often denatured and incapable of performing their intended biological functions. This aggregation indicates instability, compromising the product's shelf-life and necessitating stringent storage conditions. Additionally, the physical presence of fibrils can pose safety risks, such as causing blockages in blood vessels or tissues, which could lead to serious health issues like embolism or inflammation. Hence, minimizing fibril formation is crucial for ensuring the overall integrity and success of parenteral pharmaceutical applications.

According to WO2006051110A2, fibrillation in GLP-1 analogue solutions can be reduced by heating the solution of said peptides between 50° C. and 95° C., at a pH between 8.0 to 10.5 and then continue the heating for between 3 minutes and 180 minutes. This method would allow the fibrils to dissolve in their initial state and delay its formation. However, heating a pharmaceutical reactor to reach these temperatures requires a significant amount of energy, thereby increasing manufacturing costs and leading to higher emissions of greenhouse gases.

WO2020127476A1 discloses a method for the preparation of pharmaceutical solution comprising a GLP-1 analogue which involves heating the solution to a temperature of 26-49° C. However, heating a pharmaceutical reactor to reach these temperatures requires a significant amount of energy, thereby increasing manufacturing costs and leading to higher emissions of greenhouse gases.

WO2024061310A1 discloses GLP-1 and GIP dual receptor agonists compositions, wherein the buffer is inorganic (e.g. hydrogen phosphate) or alternatively the buffer is a citrate buffer. One potential drawback of citrate buffer is its pH range limitation. Citrate has three pKa values (≈3.1, ≈4.8, and ≈6.4), making it most effective as a buffer in the acidic to mildly neutral range (pH 3-6.5). That said, commercial tirzepatide formulations are in the pH range of 7.0-7.5, the reason why it is not optimal to use it a citrate buffer for a tirzepatide-containing pharmaceutical formulation.

Commercial trizepatide compositions need to be stored under refrigerated conditions (i.e. 2° C.-8° C.), and are only stable at room temperature for 21 days.

Therefore, there is an unresolved need to formulate GLP-1 and GIP dual receptor agonists compositions with improved stability. The pharmaceutical composition disclosed herein is directed towards addressing the shortcomings and challenges outlined above.

### SUMMARY

Disclosed herein is a pharmaceutical composition comprising a GLP-1 and GIP dual receptor agonist wherein the buffer of the pharmaceutical composition is suitable to maintain a pH between 6.5 to 8.5 throughout the shelf life of the product while avoiding excessive degradation of the dual receptor agonist.

Inorganic buffers (e.g. sodium phosphate dibasic) are used in commercial products comprising GLP-1 receptor agonists such as liraglutide (Victoza®), semaglutide (Ozempic®, Wegovy®) and also in those commercial products comprising GLP-1 and GIP dual receptor agonists, such as tirzepatide (Mounjaro®, Zepbound®).

Inorganic buffers are widely used in pharmaceutical compositions comprising the mentioned agonists due to its well-established safety profile and relatively low interaction with the active ingredients. However, the inventors have surprisingly found that stable pharmaceutical formulations disclosed herein comprising a GLP-1 and GIP dual receptor agonist prepared with a buffer other than an inorganic buffer show improved stability.

In addition, this disclosure relates to a method of manufacturing of the pharmaceutical composition disclosed herein, which is more energy efficient and confers a lower impurity formation to the manufactured product.

Each of the aspects and embodiments disclosed herein may be combined with one or more aspects and embodiments, as appropriate.

A first aspect relates to a pharmaceutical composition comprising a GLP-1 and GIP dual receptor agonist and at least one non-inorganic buffer having a pKa of at least 6.6 or above (e.g., a pKa of about 6.6 to about 8.6).

The pharmaceutical compositions disclosed herein have lower impurity content over time and less individual and total impurities than pharmaceutical solutions comprising the mentioned dual receptor agonists and inorganic buffers.

A second aspect relates to a method for the preparation of the pharmaceutical composition of the first aspect comprises the following steps:
i) preparing a solution comprising the tonicity modifier, the buffering agent, the GLP-1 and GIP dual receptor agonist and water for injection; and
ii) adjusting the pH of the final solution between 6.5-8.5, or alternatively, between 6.5-7.5, with a pH adjuster.

In a third aspect, a cartridge comprising the pharmaceutical composition of the first aspect or the pharmaceutical composition prepared according the second aspect is disclosed.

### DETAILED DESCRIPTION

As used in the present disclosure, the following words, phrases, and symbols are generally intended to have the meanings as set forth below, except to the extent that the context in which they are used indicates otherwise.

All percentages are expressed by weight (w/w) and as used herein are referred to the total weight of the composition, unless specifically noted otherwise.

The term “active ingredient” as used herein refers to a therapeutically active compound, as well as any pharmaceutically acceptable hydrates and solvates of the compound.

The term “buffer” as used herein refers to a chemical compound in a pharmaceutical composition that reduces the tendency of pH of the composition to change over time as would otherwise occur due to chemical reactions.

The term “inorganic buffer” as used herein refers to a buffering agent in a pharmaceutical composition that is derived from an inorganic acid or its conjugate base and functions to maintain a stable pH by resisting changes due to acid-base reactions. Non-limiting examples of inorganic buffers include phosphate buffers (e.g., sodium phosphate, potassium phosphate), carbonate buffers (e.g., sodium bicarbonate, potassium bicarbonate), and borate buffers (e.g., sodium borate, boric acid).

The term “non-inorganic buffer” as used herein refers to any buffering agent that is not encompassed within the definition of an inorganic buffer. Non-inorganic buffers include, but are not limited to, organic acid-base buffers (e.g., citrate, acetate, lactate), zwitterionic buffers (e.g., HEPES (4-(2-hydroxyethyl)-1-piperazineethanesulfonic acid), MOPS (3-(N-morpholino)propanesulfonic acid), PIPES (piperazine-N,N′-bis (2-ethanesulfonic acid)), BES (N,N-Bis(2-hydroxyethyl)-2-aminoethanesulfonic acid), TES (N-[Tris(hydroxymethyl)methyl]-2-aminoethanesulfonic acid), TAPS ([tris(hydroxymethyl)methylamino]propanesulfonic acid), ACES (N-(2-acetamido)-2-aminoethanesulfonic acid), MOPSO (2-Hydroxy-3-(morpholin-4-yl)propane-1-sulfonic acid), DIPSO (3-(N,N-Bis[2-hydroxyethyl]amino)-2-hydroxypropanesulfonic acid), TAPSO (3-[N-Tris(hydroxymethyl)methylamino]-2-hydroxypropanesulfonic acid), POPSO (piperazine-N,N′-bis(2-hydroxypropanesulfonic acid), HEPPSO ((2-Hydroxyethyl)-piperazine-N-2-hydroxypropanesulfonic acid), HEPPS (4-(2-Hydroxyethyl)-1-piperazinepropanesulfonic acid)), and amine-based buffers (e.g., Tris (tris(hydroxymethyl)aminomethane), Bicine (N,N-bis(2-hydroxyethyl)glycine), Tricine (N-tris(hydroxymethyl)methylglycine), and Bis-Tris (2,2-bis(hydroxymethyl)-2,2′,2″-nitrilotriethanol), cholamine chloride, triethanolamine, glycinamide). These buffers function to maintain pH stability in pharmaceutical compositions by resisting changes due to acid-base reactions.

The term “organic acid-base buffer” as used herein refers to a non-inorganic buffering agent derived from an organic acid and its conjugate base, or an organic base and its conjugate acid. These buffers function by reversible protonation and deprotonation to resist pH fluctuations, typically in the acidic to mildly neutral pH range.

The term “organic amine-based buffer” as used herein refers to a non-inorganic buffering agent that contains one or more amine (—NH₂) functional groups and is derived from an organic compound. These buffers function to resist pH fluctuations by reversible protonation and deprotonation of the amine group, typically in the neutral to alkaline pH range. Non-limiting examples of organic amine-based buffers include Tris (pKa≈8.3), Bicine (pKa≈8.4), Tricine (pKa≈8.2), and Bis-Tris (pKa≈6.5), cholamine chloride (pKa≈7.1), triethanolamine (pKa≈8), glycinamide (pKa≈8.2), and glycylglycine (pKa≈8.2), or a combination thereof.

The term “zwitterionic buffer” as used herein refers to a non-inorganic buffering agent that contains both a positively charged (cationic) and a negatively charged (anionic) functional groups at a given pH, allowing it to resist pH changes effectively while maintaining overall electrical neutrality. Zwitterionic buffers are particularly useful in biological and pharmaceutical compositions due to their minimal interaction with biomolecules and metal ions. Good, 472. Non-limiting examples of zwitterionic buffers include, for example, HEPES (pKa≈7.5), MOPS (pKa≈7.2), PIPES (pKa≈6.8), TES (pKa≈7.4), TAPS (pKa≈8.4), ACES (pKa≈6.9), MOPSO (pKa≈7), DIPSO (pKa≈7.6), TAPSO (pKa≈7.6), POPSO (pKa≈7.9), HEPPSO (pKa≈7.9), HEPPS (pKa≈8.1), or a combination thereof.

The term “pH adjuster” as used herein refers to pharmaceutically acceptable excipients which are added to the solution of the active agent to adjust the pH to a certain value. Such pH adjusters can be alkaline or acid agents and may comprise inorganic salts as well as organic acids or salts of organic acids. Examples of pH adjusters are HCl or NaOH.

The term “pharmaceutically acceptable” as used herein indicates that the substance or composition must be compatible chemically and/or toxicologically, with the other ingredients in the composition, and/or the mammal being treated therewith.

The term “pharmaceutical composition” as used herein means a product comprising an active compound or a salt thereof together with pharmaceutical excipients such as buffer, preservative and tonicity modifier, said pharmaceutical composition being useful for treating, preventing or reducing the severity of a disease or disorder by administration of said pharmaceutical composition to a person. Thus, a pharmaceutical composition is also known in the art as a pharmaceutical formulation.

The term “pKa” as used herein refers to the negative base-10 logarithm of the acid

dissociation constant (Ka) of a weak acid, representing the pH at which the weak acid exists in equilibrium at an equimolar amount with its conjugate base. The pKa value of a buffer determines its optimal pH range for resisting pH fluctuations in a pharmaceutical composition. Lower pKa values correspond to stronger acids, while higher pKa values indicate weaker acids. Non-limiting examples include the pKa of citric acid (≈3.1, ≈4.8, ≈6.4), phosphate buffer ≈7.2), HEPES (≈7.5), and Tris (≈8.1).

The term “preservative” as used herein refers to a chemical compound which is added to a pharmaceutical composition to prevent or delay microbial activity (growth and metabolism). Examples of pharmaceutically acceptable preservatives are phenol, m-cresol, methyl p-hydroxybenzoate, propyl p-hydroxybenzoate, benzoic acid, benzyl alcohol, benzyl benzoate, 2-phenoxyethanol, butyl p-hydroxybenzoate, 2-phenylethanol, benzyl alcohol, chlorobutanol, acetone sodium bisulfite, benzalkonium chloride, benzethonium chloride, thiomerosal, or a combination thereof.

The term “stable” as used herein refers to any pharmaceutical composition comprising the active ingredient having a sufficient physical and chemical stability to allow storage under any of the general storage conditions as defined by ICH Q1A (R2).

The term “tonicity modifier” as used refers to a chemical compound in a pharmaceutical composition that serves to modify the osmolality of the pharmaceutical composition so that its osmolality approximates the osmolality of human plasma. The tonicity modifier is also known in the art as “isotonicity agent”. Isotonicity agents include at least one of mannitol, sorbitol, lactose, propylene glycol dextrose, trehalose, sodium chloride, potassium chloride, glycerol, glycerin, etc.

The quantitative determination of tirzepatide by UPLC analysis was carried out using ACQUITY UPLC H-Class PLUS system, Aeris Peptide XB C18 100 Å 4.6 mm×250 mm, 3.6 μm column was used. A Segurity Guard ULTRA cartridge, UHPLC C18-Peptide 4.6 mm ID pre-column was used. The apparatus was equipped with a manual injector and UV detector. The injection valve was a Rheodyne with a capacity of 20 μL. Mobile phase A (0.5% TFA in Water:Methanol (95:5, v/v % ) and Mobile phase B (0.5% TFA in Acetonitrile:Methanol:Water (90:5:5, v/v/v %) with 6.0 mL NH3 30%) were used at a ratio 40:60. As a diluent, 0.025% v/v Ammonia in water was used. The mobile phases were filtered through a 0.45 um membrane filter and sonicated before use. It was pumped through the column at a flow rate of 0.8 mL/min. Injection volume was 5 μL and the column was maintained at 55° C. The detection was monitored at 220 nm and the run time was set as 35 minutes. The amount of tirzepatide in the samples was determined by comparison with appropriate external standard curves obtained applying the least square linear regression analysis.

The quantitative determination of tirzepatide impurities by UPLC was carried out using an ACQUITY UPLC H-Class PLUS system, Acquity UPLC Peptide CSH C18 130 Å 2.1×150 mm, 1.7 μm (Two columns connected in series with column coupler) column was used. An Acquity UPLC Peptide CSH C18 Vanguard Pre-Column 130 Å 5×2.1 mm, 1.7 μm pre-column was used. Mobile phase A (Buffer solution 4 mM:Methanol:TFA (950:50:1, v/v/v %) adjusted to pH 7.00 with Ammonia solution) and Mobile phase B (Acetonitrile:Methanol:Water:TFA (500:450:50:1, v/v/v/v %) were used at a ratio 26:74. As a diluent, 0.025% v/v Ammonia in water was used. The mobile phases were filtered through a 0.45 μm membrane filter and sonicated before use. It was pumped through the column at a flow rate of 0.2 mL/min. Injection volume was 4 μL and the column was maintained at 55° C. The detection was monitored at 215 nm and the run time was set as 145 minutes. The percentage of other impurities are calculated by the following equation:

$$\% Impurity = ( As \times Wst \times P \times Ds \times Cf ) / ( Ast \times Ws \times fdst \times C )$$

Where,
As: Impurity peak area in test solution
Ast: Tirzepatide peak area in reference solution
Wst: Weight of tirzepatide standard in Reference Solution (mg)
Ws: Weight of sample in test solution (g)
P: Purity of tirzepatide standard as is (%), into account KF determination before use (H)

$$P = P tirzepatide \times ( 100 - ( H ) ) / 100$$

fdst: Dilution factor of Reference Solution (2500)
fdS: Dilution factor of test solution (10)
Ds: Density of product (g/mL)
C: Content of tirzepatide in 1 mL, in mg (6.0)
Cf: Correction factor (1/RRF=Tirzepatide RF/Impurity RF)

The term “unknown impurity” as used herein refers to an impurity of unknown structure having a specific relative retention time (RRT or tRr) in each case. The percentage of each impurity is calculated as explained above from the results of the analysis under the UPLC conditions set forth above.

The term “analogue” as used herein referring to a peptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide.

The term “derivative” as used herein in relation to a parent peptide means a chemically modified parent protein or an analogue thereof, wherein at least one substituent is not present in the parent protein or an analogue thereof, i.e. a parent protein which has been covalently modified. Typical modifications are amides, carbohydrates, alkyl groups, acyl groups, esters, PEGylations and the like.

The term “dual GLP-1 and GIP agonist”, as used herein, refers to a class of drugs that activate both the glucagon-like peptide-1 (GLP-1) receptor and the glucose-dependent insulinotropic polypeptide (GIP) receptor. These agents mimic the actions of the endogenous incretin hormones GLP-1 and GIP, which are released by the gut in response to food intake, thereby enhancing insulin secretion, reducing blood glucose levels, and regulating energy balance. Non-limiting examples of dual GLP-1 and GIP agonists known in the art include tirzepatide, CT-388, VK2735, CT-868, and HRS9531.

All percentages, parts and ratios herein used are by weight unless specifically noted otherwise. As used herein, the term “about” refers to a range that is ±10%, ±5%, or ±1% of a value with which the term is associated.

Unless otherwise indicated, all the analysis methods are carried out according to the European Pharmacopoeia 10th edition.

The inventors have found out that a pharmaceutical composition disclosed herein has a low level of impurities.

A first aspect relates to a pharmaceutical composition comprising a GLP-1 and GIP dual receptor agonist and at least one non-inorganic buffer having a pKa of at least 6.6 or above (e.g., a pKa of about 6.6 to about 8.6). In one aspect, the pharmaceutical composition comprises tirzepatide in an amount of from about 1 mg/mL to about 30 mg/mL and all values in between, such as 2.5 mg/mL, 5 mg/mL, 10 mg/mL, 15 mg/mL, 20 mg/mL, and 25 mg/mL, where the mole ratio of the at least one non-inorganic buffer to tirzepatide ranges from about 0.1 to about 10, as well as all values in between, e.g., 2, 3, 4, 5, 6, 7, 8, and 9.

In an embodiment, the non-inorganic buffer comprises a zwitterionic buffer. In one aspect, the zwitterionic buffer comprises ACES, BES, DIPSO, HEPES, HEPPS, HEPPSO, MOPS, MOPSO, PIPES, POPSO, TAPS, TAPSO, TES, or a combination thereof. In another aspect, the zwitterionic buffer comprises HEPES.

In another embodiment, the non-inorganic buffer comprises an organic amine-based buffer. In one aspect, the organic amine-based buffer comprises Tris, Bicine, Tricine, Bis-Tris, cholamine chloride, triethanolamine, glycinamide, or a combination thereof. In another aspect, the organic amine-based buffer comprises Tris.

In another embodiment, the non-inorganic buffer is an organic acid-base buffer.

In another embodiment, the GLP-1 and GIP dual receptor agonist is tirzepatide.

In another embodiment, the pharmaceutical composition is free from an inorganic buffer. In one aspect, the pharmaceutical composition excludes (or is explicitly free from) an inorganic buffer.

In another embodiment, the pharmaceutical composition is explicitly free from (or excludes) a phosphate buffer, non-limiting examples of phsophate buffers comprise sodium phosphate buffer, potassium phosphate buffer, phosphate-buffered saline (PBS), Sørensen's phosphate buffer, Mcllvaine phosphate-citrate buffer and phosphate-carbonate buffer. In a more preferred embodiment, the pharmaceutical composition is free from (or excludes) sodium phosphate buffer.

In one embodiment, the pharmaceutical composition is explicitly free from (or excludes) citrate buffer. As used herein, ‘explicitly free from’ means that the composition does not contain any intentionally added citrate-based buffering agents, including citric acid or its salts, such as sodium citrate or potassium citrate.

In another embodiment, the pharmaceutical composition is explicitly free from (or excludes) carbonate buffer. As used herein, ‘explicitly free from’ means that the composition does not contain any intentionally added carbonate-based buffering agents, including sodium carbonate, potassium carbonate, sodium bicarbonate, or potassium bicarbonate.

In another embodiment, the pharmaceutical composition is explicitly free from (or excludes) borate buffer. As used herein, ‘explicitly free from’ means that the composition does not contain any intentionally added borate-based buffering agents, including boric acid, sodium borate, or potassium borate.

In another embodiment, the pharmaceutical composition further comprises sodium chloride and water.

In another embodiment, the pH of said pharmaceutical composition further comprising sodium chloride and water is between 6.5-8.5. In one aspect, the pH is 6.5-7.5, in another aspect, the pH is 6.8-7.3.

In an embodiment, the pharmaceutical composition has a total impurity content after 1 month storage at 25° C./60% RH is below 5%, below 4%, below 3%, or below 2%.

In an embodiment, the pH of the pharmaceutical composition after 1 month storage at 25° C./60% RH does not suffer a variation greater than 0.5 units from the initial time point.

In an embodiment, the pharmaceutical composition is to be administered parenterally. In one aspect, the route of administration is intramuscular, intravenous or subcutaneous. In another aspect, the administration route is subcutaneous.

In one embodiment, the pharmaceutical composition is indicated for the treatment, prevention, or management of metabolic disorders and conditions associated with dysregulated glucose and energy homeostasis. Such conditions include, but are not limited to, type 2 diabetes mellitus, obesity, metabolic syndrome, insulin resistance, and cardiovascular complications related to metabolic dysfunction. In certain embodiments, the pharmaceutical composition is administered to a subject in need thereof to improve glycemic control, enhance insulin sensitivity, promote weight loss, and reduce cardiovascular risk factors.

A second aspect relates to a method for the preparation of the above pharmaceutical composition which comprises the following steps:
i) preparing a solution comprising the tonicity modifier, the buffering agent, the GLP-1 and GIP dual receptor agonist and water for injection; and
ii) adjusting the pH of the final solution between 6.5-8.5, or alternatively between 6.5-7.5, with a pH adjuster.

In an embodiment, the solution of step i) is prepared by mixing all the inactive ingredients and the active ingredient directly into one unique solution. Alternatively, the solution of step i) is prepared by mixing a first solution comprising the inactive ingredients and a second solution comprising the active ingredient to create a final solution. Also alternatively, the solution of step i) is prepared by combining a first solution and a second solution to obtain a final solution.

In a further embodiment, the non-inorganic buffering agent is the buffering agent of the first aspect of the disclosed pharmaceutical composition.

In a further embodiment, the dual receptor agonist is the active ingredient of the first aspect of the disclosed pharmaceutical composition.

In a further embodiment, the final solution is prepared at a temperature between 2-15° C., 2-10° C., 2-8° C., or 3-6° C. Cooling a pharmaceutical reactor to 2-15° C. is generally more energy efficient than heating it to 50° C., as refrigeration systems can remove heat with a lower energy input compared to direct heating methods. Additionally, heat loss to the environment naturally aids cooling, whereas heating requires continuous energy input to maintain high temperatures.

In an embodiment, the method may comprise bubbling nitrogen gas in any of the first, second, and/or final solutions, e.g., until the dissolved oxygen content is <2.0 ppm. It has been found out that bubbling nitrogen gas helps to avoid the formation of fibrils, particularly when the dissolved oxygen content is kept below 2.0 ppm.

In an embodiment, the method may comprise filtering the final solution through a 0.2 μm pore size filter. Filtering the final solution through a 0.2 μm pore size filter delays the formation of fibrils.

In an embodiment, the tonicity modifier may be selected from the group consisting of mannitol, sorbitol, lactose, propylene glycol, dextrose, trehalose, sodium chloride, potassium chloride, glycerol, glycerin or mixtures thereof. In one aspect, the tonicity modifier is sodium chloride.

In an embodiment, the method comprises filling aseptically the final solution into cartridges flushed with nitrogen gas. The nitrogen gas flushed in the cartridges delays the appearance of fibrils. In an embodiment, no air bubbles remain inside the cartridges. The inventors have surprisingly found that when air bubbles remain in the cartridge, even if nitrogen is flushed, the concentration of fibrils is higher in the pharmaceutical composition.

A third aspect relates to a cartridge comprising the pharmaceutical composition of the first aspect or the pharmaceutical composition prepared according the second aspect.

Further embodiments disclosed herein can be found in the following numbered clauses:
Clause 1. A pharmaceutical composition comprising a GLP-1 and GIP dual receptor agonist and at least one non-inorganic buffer having a pKa of at least 6.6 or above (e.g., a pKa of about 6.6 to about 8.6).
Clause 2. The pharmaceutical composition of clause 1 wherein the buffer is a zwitterionic buffer.
Clause 3. The pharmaceutical composition of clause 1 wherein the buffer is an organic amine-based buffer.
Clause 4. The pharmaceutical composition of clause 2, wherein the buffer is ACES, BES, DIPSO, HEPES, HEPPS, HEPPSO, MOPS, MOPSO, PIPES, POPSO, TAPS, TAPSO, TES, or a combination thereof.
Clause 5. The pharmaceutical composition of clause 3, wherein the buffer is Tris, Bicine, Tricine, Bis-Tris, cholamine chloride, triethanolamine, glycinamide, or a combination thereof.
Clause 6. The pharmaceutical composition according to any of the preceding clauses, wherein the dual receptor agonist is tirzepatide.
Clause 7. The pharmaceutical composition according to any of the preceding clauses, wherein said composition is free from sodium phosphate buffer.
Clause 8. The pharmaceutical composition according to any of the preceding clauses, further comprising sodium chloride and water, and wherein the pH of said composition ranges between 6.5 to 7.5.
Clause 9. The pharmaceutical composition according to any of the preceding clauses, wherein the total impurity content after 1 month storage at 25° C./60% RH is below 5%, below 4%, below 3%, and below 2%.
Clause 10. The pharmaceutical composition according to any of the preceding clauses, wherein the pH of the composition after 1 month storage at 25° C./60% RH has not suffered a variation greater than 0.5 units from the initial time point.
Clause 11. The pharmaceutical composition according to any of the preceding clauses, wherein said composition is for sub-cutaneous administration.
Clause 12. A method for the preparation of a pharmaceutical composition comprising a GLP-1 and GIP dual receptor agonist, a tonicity modifier and a non-inorganic buffering agent having a pKa of at least 6.6 or above (e.g., a pKa of about 6.6 to about 8.6) which comprises the following steps:

preparing a solution comprising the tonicity modifier, the buffering agent, the GLP-1 and GIP dual receptor agonist and water for injection; and adjusting the pH of the final solution between 6.5-8.5, or alternatively between 6.5-7.5, with a pH adjuster.
Clause 13. The method according to the preceding clause wherein the solution is prepared at a temperature between 2-15° C., 2-10° C., 2-8° C., or 3-6° C.
Clause 14. The method according to the preceding clause wherein the dual receptor agonist is tirzepatide.
Clause 15. The method according to any of the three preceding clauses, wherein the buffering agent is selected from the group consisting of HEPES, tris, and a combination thereof.
Clause 16. The method according to any of the four preceding clauses wherein the tonicity modifier agent is sodium chloride.
Clause 17. A method according to any of the clauses 13-16, wherein the final solution temperature does not exceed 15° C.
Clause 18. A method according to any of the clauses 13-16, wherein the final solution temperature does not exceed 14° C.
Clause 19. A method according to any of the clauses 13-16, wherein the final solution temperature does not exceed 13° C.
Clause 20. A method according to any of the clauses 13-16, wherein the final solution temperature does not exceed 12° C.
Clause 21. A method according to any of the clauses 13-16, wherein the final solution temperature does not exceed 11° C.
Clause 22. A method according to any of the clauses 13-16, wherein the final solution temperature does not exceed 10° C.
Clause 23. A method according to any of the clauses 13-16, wherein the final solution temperature does not exceed 9° C.
Clause 24. A method according to any of the clauses 13-16, wherein the final solution temperature does not exceed 8° C.
Clause 25. A cartridge comprising the pharmaceutical composition according to any of the clauses 1-11 or the compositions directly obtained by the method according to any of the clauses 12-24.
Clause 26. A pharmaceutical composition comprising tirzepatide and HEPES buffer.
Clause 27. A pharmaceutical composition according to the preceding clause further comprising sodium chloride and water, and wherein the pH of said composition ranges between 6.5 to 7.5.
Clause 28. The pharmaceutical composition according to any of the two preceding clauses wherein said composition is free from citrate buffer and free from phosphate buffer.
Clause 29. The pharmaceutical composition according to any of the three preceding clauses with the proviso that the composition is not a hydrogel composition.
Clause 30. The pharmaceutical composition according to any of the four preceding clauses wherein the composition is a solution for sub-cutaneous administration.
Clause 31. The pharmaceutical composition according to any of the five preceding clauses wherein said composition is free from hydrogel forming polymers.
Clause 32. A pharmaceutical composition comprising a tirzepatide and tris.
Clause 33. A pharmaceutical composition according to the preceding clause further comprising sodium chloride and water, and wherein the pH of said composition ranges between 6.5 to 7.5.
Clause 34. The pharmaceutical composition according to any of the two preceding clauses wherein said composition is free from citrate buffer and free from phosphate buffer.
Clause 35. The pharmaceutical composition according to any of the three preceding clauses with the proviso that the composition is not a hydrogel composition.
Clause 36. The pharmaceutical composition according to any of the four preceding clauses wherein the composition is a solution for sub-cutaneous administration.
Clause 37. The pharmaceutical composition according to any of the five preceding clauses wherein said composition is free from hydrogel forming polymers.
Clause 38. A pharmaceutical composition according to any of the clauses 1-11 with the proviso that the composition is not a hydrogel composition.
Clause 39. A pharmaceutical composition according to the preceding clause wherein the composition is a solution for sub-cutaneous administration.
Clause 40. A pharmaceutical composition according to any of the two preceding clauses wherein the composition is free from hydrogel forming polymers.
Clause 41. A pharmaceutical composition according to any of the clauses 1-11 or 38-40 wherein the dual receptor agonist concentration is 5 mg/mL.
Clause 42. A pharmaceutical composition according to any of the clauses 1-11 or 38-41 comprising sodium chloride as tonicity agent at a molar concentration of 0.14M.
Clause 43. A pharmaceutical composition according to any of the clauses 1-11 or 38-42 wherein the oxygen content is <2.0 ppm.
Clause 44. A pharmaceutical composition according to any of the clauses 1-11 or 38-42 having a fibril content of not more than about 0.04 μM after storage for 1-month at 30° C. and 65% relative humidity, where the fibril content is measured by the Thioflavin T Fibrillation Estimation Assay.

### EXAMPLES

### Example 1. Preparation of Pharmaceutical Compositions Comprising Tirzepatide

| | | | Comp. example |
|---|---|---|---|
| Component | GLN-1 | GLN-2 | 1 |
| Tirzepatide | 5 | 5 | 5 |
| Sodium Chloride | 8.2 | 8.2 | 8.2 |
| Sodium Phosphate | — | — | 1.4 |
| dibasic heptahydrate | | | |
| Tris | — | 0.63 | — |
| HEPES | 1.19 | — | — |
| HCl/NaOH | q.s. to | q.s. to | q.s. to |
| | pH 7 | pH 7 | pH 7 |
| Water for injection | q.s. to | q.s. to | q.s. to |
| | 1 mL | 1 mL | 1 mL |
| Nitrogen | q.s | q.s | q.s |

The amount of the different components is expressed as mg/mL.

The GLN-1, GLN-2 and Comp. Example 1 formulations were manufactured by preparing a first solution comprising sodium chloride, the corresponding buffering agent, and water for injection. In another container, a second solution comprising tirzepatide and water for injection was prepared. Then, a final solution was prepared by mixing the first and the second solution. The first, second and final solutions were prepared at a temperature between 2-8° C. Nitrogen gas was bubbled in the first, second and final solution, to maintain the dissolved oxygen content below 2.0 ppm. Then, the pH of the final solution was adjusted to 7 with HCl and/or NaOH. Eventually, the final solution was filtered through a 0.2 μm filter.

### Example 2. Impurity Content

All formulations obtained in Example 1 were analysed by UPLC as described above for impurity content at two different times, freshly prepared (t=0) and at 1 month stored at 25° C./60% RH. The following results were obtained:

| | GLN-1 | | GLN-2 | | Comp. Ex. 1 | |
|---|---|---|---|---|---|---|
| | t = 0 | t = 1 | t = 0 | t = 1 | t = 0 | t = 1 |
| TZP-L-beta-Asp15 [Impurity | ND | 0.05 | ND | 0.03 | ND | ND |
| A] (t_{Rr} = 0.86) | | | | | | |
| Unknown Impurity (t_{Rr} = | 0.04 | 0.03 | 0.03 | 0.01 | 0.03 | 0.01 |
| 0.97) | | | | | | |
| D-Ser32 + 33 [Impurity C] | 0.02 | 0.05 | 0.02 | 0.05 | 0.02 | 0.04 |
| (t_{Rr} = 1.03) | | | | | | |
| TZP-L-beta-Asp9 [Impurity | 0.04 | 0.15 | 0.04 | 0.14 | 0.05 | 0.14 |
| D] (t_{Rr} = 1.05) | | | | | | |
| Unknown Impurity (t_{Rr} = | ND | 0.02 | ND | 0.02 | ND | 0.43 |
| 0.32) | | | | | | |
| Unknown Impurity (t_{Rr} = | ND | 0.06 | ND | 0.12 | ND | ND |
| 0.88) | | | | | | |
| Unknown Impurity (t_{Rr} = | ND | 0.80 | ND | 1.08 | ND | 1.90 |
| 0.90) | | | | | | |
| Unknown Impurity (t_{Rr} = | ND | 0.05 | ND | ND | ND | ND |
| 0.92) | | | | | | |
| Unknown Impurity (t_{Rr} = | 0.02 | 0.03 | 0.03 | 0.02 | 0.03 | 0.06 |
| 0.95) | | | | | | |
| Total impurities (%) | 0.12 | 1.24 | 0.12 | 1.47 | 0.13 | 2.58 |

All amounts of impurities are expressed as a percentage, “ND” corresponds to not detected. As it can be seen, the amount of the majority of the impurities over time is lower in GLN-1 and GLN-2, as well as the percentage of total impurities.

### Example 3. Thioflavin T (ThT) Fibrillation Estimation Assay

Low physical stability of a peptide may lead to amyloid fibril formation, which is observed as well-ordered, thread-like macromolecular structures in the sample eventually resulting in gel formation. This has traditionally been measured by visual inspection of the sample. However, that kind of measurement is very subjective and depends on the observer. Therefore, the application of a small molecule indicator test is much more advantageous. Thioflavin T (ThT) is such a test and has a distinct fluorescence signature when binding to fibrils.

To minimise costs related to the API and being able to estimate the fibrils in the compositions (at 5 mg/mL) by ThT fibrillation assay, Bovine serum albumin (BSA) was used for the standard curve of the assay, since this protein has similar tendency to fibril formation as tirzepatide. To this aim, a BSA standard curve was prepared from 0.188 to 0.003 mM and a Thioflavin-T working solution at 50 uM. After preparation of standard concentration and one blank (PBS), the BSA standard samples at 80° C. for 30 min and then cool at room temperature for fibril generation.

### Preparation of the Samples

Preparation of standard Blank: Add 50 μL of heated and cooled PBS into 96 well plate in duplicate.
Preparation of Standard: Add 50 μL of heated and cooled BSA (0.188 mM-0.003 mM) each into 96 well plate in duplicate.
Preparation of Test samples Blank: Add 50 μL of Milli-Q water into 96 well plate in duplicate.
Preparation of Test samples: Add 50 μL Test samples (50 μM) into 96 well plate in duplicate. Then read the plate as per method created in Micro plate reader.

After addition of all blanks, standard curve concentrations and test sample concentrations in the plate, added 150 μL of thioflavin-T working solution (50 μM) into each well and mixed gently by pipetting. Read the plate as per the following microplate reader conditions:

(Read Mode: Fluorescence; excitation wavelength: 440 nm; emission wavelength: 482 nm; bandwidth excitation: 9 nm; bandwidth emission: 15 nm; plate type: 96 well standard opaque; plate height: 14.6 mm; plate Shake: 60 seconds, orbital, medium (before first read); PMT and optics: 6 flashes/read; Read from: Top; Read height: 1.00 mm)

### Calculations and Results

Plotted the standard curve with the blank subtracted values of standards on Y-axis and their concentrations on X-axis.

Derived slope (Mstd), intercept (Cstd) and regression (R2) from the straight-line equation.

The following compositions were measured according to the above ThT fibrillation assay:

GLN-1, GLN-2 and Comp. Ex. 1 were prepared as described in Example 1. Comp. Ex. 2 was prepared as Comp. Ex. 1 but the process was carried by heating the solutions during the whole process at 60° C.

After preparation, the samples were stored for 1 month at 30° C./Relative Humidity 65% and analysed. Afterwards, samples were analysed according to the ThT fibrillation assay detailed before and the following results were obtained:

| Composition | Fibrils content (μM) |
|---|---|
| GLN-1 | 0.0255 |
| GLN-2 | 0.0392 |
| Comp. Ex. 1 | 0.0282 |
| Comp. Ex. 2 | 0.1459 |

### CITED INFORMATION

European Pharmacopoeia 10th edition, 2019.
ICH Harmonized Tripartite Guideline: Stability Testing of New Drug Substances and Products, Feb. 6, 2003 (“ICH Q1A (R2)”).
EP 3 810 201 B1, GIP/GLP1 agonist compositions, published on Jun. 7, 2023, to Corvari et al., of Eli Lilly & Co. (related to U.S. Pat. No. 11,357,820B2).
Good et al., Hydrogen ion buffers for biological research, Biochemistry (1966) 5(2): 467-477.
Mounjaro® (tirzepatide) Injection, May 13, 2022 Prescribing Information.
U.S. Pat. No. 8,748,376 B2, Stable Formulations of Peptides, issued on Jun. 10, 2014, to Ludvigsen et al. of Novo Nordisk A/S (related to WO 2006/051110 A2).
U.S. Pat. No. 11,357,820 B2, GIP/GLP1 agonist compositions, issued on Jun. 14, 2022 to Corvari et al., of Eli Lilly & Co. (related to EP3810201B1).
WO 2006/051110 A2, Stable Formulations of Peptides, published on May 18, 2006, to Ludvigsen et al. of Novo Nordisk A/S (related to U.S. Pat. No. 8,748,376B2).
WO 2020/127476 A1, Pharmaceutical composition comprising GLP-1 Analogue, published on Jun. 25, 2020, to Roszman et al. of KRKA, D. D., Novo Mesto.
WO 2024/061310 A1, Dual GLP-1 and GIP receptor agonist pharmaceutical composition and use thereof, published on 28 Mar. 2024 to Li et al. of Brightgene Bio-medical Technology Co., Ltd., et al.
WO 2024/006662 A1, Tirzepatide compositions and use, published on Jan. 4, 2024 to Koenig et al. of Eli Lilly & Co.
Zepbound® (tirzepatide) Injection, Nov. 8, 2023 Prescribing Information.

The cited information is incorporated by reference to the extent necessary to understand embodiments and/or aspects disclosed herein. The subject matter of European patent application EP24382587, filed on May 31, 2024; European patent application EP25382104, filed on Feb. 7, 2025; and U.S. patent application Ser. No. 19/019,721, filed on Jan. 14, 2025, is incorporated by reference in its entirety except that if a conflict exists between the incorporated subject matter and the subject matter disclosed herein, then the subject matter herein controls.

1. A pharmaceutical composition comprising a GLP-1 and GIP dual receptor agonist and at least one non-inorganic buffer having a pKa of at least about 6.6.

2. The pharmaceutical composition of claim 1, wherein the non-inorganic buffer comprises a zwitterionic buffer.

3. The pharmaceutical composition of claim 1, wherein the non-inorganic buffer comprises an organic amine-based buffer.

4. The pharmaceutical composition of claim 2, wherein the zwitterionic buffer comprises ACES, BES, DIPSO, HEPES, HEPPS, HEPPSO, MOPS, MOPSO, PIPES, POPSO, TAPS, TAPSO, TES, or a combination thereof.

5. The pharmaceutical composition of claim 1, wherein the at least one non-inorganic buffer comprises HEPES, tris, or a combination thereof.

6. The pharmaceutical composition of claim 1, wherein the at least one non-inorganic buffer comprises HEPES.

7. The pharmaceutical composition of claim 1, wherein the at least one non-inorganic buffer comprises tris.

8. The pharmaceutical composition of claim 3, wherein the organic amine-based buffer comprises Tris, Bicine, Tricine, Bis-Tris, cholamine chloride, triethanolamine, glycinamide, or a combination thereof.

9. The pharmaceutical composition of claim 1, wherein the dual receptor agonist is tirzepatide.

10. The pharmaceutical composition of claim 9, wherein the tirzepatide is present in an amount of from about 1 mg/mL to about 30 mg/mL, and wherein the mole ratio of the at least one non-inorganic buffer to tirzepatide ranges from about 0.1 to about 10.

11. The pharmaceutical composition of claim 1, wherein said composition is free from sodium phosphate buffer.

12. The pharmaceutical composition of claim 1, further comprising sodium chloride and water, and wherein the pH of said composition ranges between 6.5 to 7.5.

13. The pharmaceutical composition of claim 1, wherein the total impurity content after 1 month storage at 25° C./60% RH is below 5%.

14. The pharmaceutical composition of claim 1, wherein the pH of the composition after 1 month storage at 25° C./60% RH has not suffered a variation greater than 0.5 units from the initial time point.

15. The pharmaceutical composition of claim 1, having a fibril content of not more than about 0.04 μM after storage for 1-month at 30° C. and 65% relative humidity, where the fibril content is measured by the Thioflavin T Fibrillation Estimation Assay.

16. The pharmaceutical composition of claim 1, wherein said composition is for subcutaneous administration.

17. A method for the preparation of the pharmaceutical composition of claim 1 which comprises:
i) preparing a solution comprising a tonicity modifier, the buffer, the GLP-1 and GIP dual receptor agonist, and water for injection; and
ii) adjusting the pH of the final solution between 6.5-8.5 with a pH adjuster.

18. The method of claim 17, wherein the dual receptor agonist is tirzepatide.

19. The method of claim 17, wherein the buffering agent comprises HEPES, tris, or a combination thereof.

20. A cartridge comprising the pharmaceutical composition of claim 1.
